(19) United States
(12) Patent Application Publication
(10) Pub. No.: US 2006/0064112 A1
(43) Pub. Date: Mar. 23, 2006

(54) Ocular device applicator

(76) Inventor: Edward Perez, Redwood City, CA (US)

Correspondence Address: MORRISON & FOERSTER LLP, 755 PAGE MILL RD, PALO ALTO, CA, 94304-1018, US

(21) Appl. No.: 10/937,206

(22) Filed: Sep. 8, 2004

(51) Int. Cl. A61F 9/00

(52) U.S. Cl. 606/107

(57) ABSTRACT

An ocular device applicator for inserting an ocular device beneath a corneal epithelium. The ocular device applicator places an ocular device onto a cornea that has been at least partially delaminated. The ocular device applicator comprises an ocular device holder and a manipulator. In some versions an ocular device may be secured in and/or released from the device by force applied in the holder, or by a releasable adhesive.

### FIELD

The described devices and methods are useful in the field of ophthalmology. Described herein are applicators and methods of using applicators for introducing an ocular device beneath a corneal epithelium. The described devices and methods for using them involve placing an ocular device onto a corneal surface which has been at least partially delaminated, for example, leaving a flap or pocket of the corneal epithelium attached to the eye that may later be replaced over an inserted ocular device. The applicator is configured to position and deposit an ocular device atop a partially delaminated cornea, e.g., between the epithelium and the corneal stroma (Bowman's membrane) in the region of the lamina lucida. The devices and methods described herein may be used as part of an ocular therapy including ocular corrective surgery and laser eye corrective surgery.

### BACKGROUND

Refractive surgery refers to a set of surgical procedures that change the native optical or focusing power of the eye. The result of these procedures often alleviates the need for glasses or contact lenses that an individual might otherwise be dependent on for clear sight. The majority of the focusing power in the human eye is dictated by the curvature of the air-liquid interface, where there is the greatest change in the index of refraction. This curved interface is the outer surface of the cornea. The refractive power of this interface accounts for approximately 70% of the total magnification of the eye. Light rays making up seen images pass through the cornea, the anterior chamber, the crystalline lens, and the vitreous humor before being focused on the retina to form an image. It is the magnifying power of this curved, air-corneal interface that provided the field of refractive surgery with the opportunity to surgically correct visual deficiencies.

Early refractive surgical procedures corrected nearsightedness by flattening the curvature of the cornea. The first largely successful procedure was called radial keratotomy (RK). RK was widely used during the 1970's and early 1980's where radially oriented incisions were made in the periphery of the cornea. These incisions reformed the peripheral cornea by causing it to bow outwards, consequently flattening the central optical zone of the cornea. This was fairly easy and thus, popular, but it rarely did more than lessen one's dependency on glasses or contract lenses.

A largely flawed and failed procedure called epikeratophakia was developed in the era of RK. It is now essentially an academic anomaly. Epikeratophakia provided a new curvature to the outer curvature of the cornea by grafting onto the cornea a thin layer of preserved corneal tissue. The processed corneal tissue is freeze-dried and during the process of freeze drying, the cornea is also ground to a specific curvature. The resulting lens was placed into the eye surgically. An annular 360° incision was placed into the cornea after completely removing the epithelium from where the epikeratophakic lens would sit. The perimeter of this lens would be inserted into the annular incision and held in place by a running suture. There were several problems with epikeratophakia: 1) the lenses remained cloudy until host stromal fibroblasts colonized the lens, which colonization possibly could take several months; 2) until migrating epithelium could grow over the incision site onto the surface of the lens, the interrupted epithelium was a nidus for infection; and 3) epithelium healing onto the surgical site sometimes moved into the space between the lens and the host cornea. Currently, epikeratophakia is limited in its use. It is now used in pediatric aphakic patients who are unable to tolerate very steep contact lenses.

Around the mid 1990's procedures that sculpt the cornea with lasers were sufficiently successful that they began to replace radial keratotomy. The first generation of laser ablation of the cornea was called photorefractive keratectomy (PRK). In PRK, an ablative laser (e.g., an excimer laser) is focused on the cornea to sculpt a new curvature into the surface. In PRK, the epithelium is destroyed when achieving a new outer surface curve. Over the ensuing post-operative days, the epithelium has to grow or heal back into place. This epithelial healing phase was problematic for most patients since the epithelially denuded and ablated cornea was painful. It is also initially difficult to see following PRK, and this “recuperative time” can last from days to a week or more.

A subsequent variation of PRK corneal laser ablation, LASIK, has become very popular. The LASIK procedure, also known as laser in situ keratomileusis, is currently synonymous in the public mind with laser vision correction. In LASIK, an outer portion (or chord-like lens-shaped portion) of the cornea (80 to 150 microns thick) is surgically cut from the corneal surface. This is performed by a device called a microkeratome. The microkeratome cuts a circular flap from the surface of the cornea, leaving the flap comprising both epithelial and corneal tissue hinged at one edge. This flap is reflected back and an ablative (excimer) laser is used to remove or to reform a portion of the exposed surgical bed. The flap is laid back into place. When this flap is laid back into place, the cornea achieves a new curvature because the flap conforms to the laser-modified surface. In this procedure, epithelial cells are not removed or harmed. The epithelial cells have simply been incised at the edge of this flap. When the flap is placed back onto the corneal bed, the epithelium heals back at the incision site. There is essentially no recuperative time and the results are almost immediate. Because there is very little surgical time (15 minutes for each eye) and because there are lasting and very accurate results, LASIK is currently considered the premier manner of performing refractive surgery.

The newest technique being evaluated in high volume refractive surgical practices and in some academic centers is a procedure called Laser Assisted Subepithelial Keratomileusis (LASEK). In LASEK, a “flap” is made of only epithelium. This layer of epithelium is lifted off the cornea in a manner similar to LASIK but using an ethanolic wash. The ablative laser is focused just on the surface of the denuded cornea (in the same manner as was done with PRK). However, this epithelial flap is left intact, i.e., the epithelium physical structure is not destroyed although cellular viability is largely destroyed. It is simply rolled back into place after formation of the re-curved anterior portion of the cornea, resulting in much less recuperative time than with PRK. Current methods of LASEK are not as good as LASIK but the results are better than with PRK.

The corneal epithelium is a multilayered epithelial structure typically about 50 μm in thickness. It is non-cornified. The outer cells are living, although they are squamous in nature. The basal epithelial cells are cuboidal and sit on the stromal surface on a structure known as Bowman's membrane. The basal cell layer is typically about 1 mil thick (0.001″). The basal cells produce the same keratins that are produced in the integument, i.e., skin. The basal epithelial cells express keratins 5 and 14 and have the potential to differentiate into the squamous epithelial cells of the corneal epithelium that produce keratins 6 and 9. The corneal epithelium has a number of important properties: 1) it is clear; 2) it is impermeable; 3) it is a barrier to external agents; and 4) it is a highly innervated organ. Nerves from the cornea directly feed into the epithelium, and thus, defects of this organ produce pain.

Epithelial cells are attached side-to-side by transmembrane molecules called desmosomes. Another transmembrane protein, the hemidesmosome, connects to collagen type 7 and is present on the basolateral surface of basal epithelial cells. Hemidesmosomes anchor epithelium to the underlying collagenous portion of the stroma. The junction between the epithelium and corneal stroma is referred to as basement membrane zone (BMZ).

When LASEK is performed, a physical well is placed or formed on the epithelium and filled with a selection of 20 percent ethanol and balanced salt solution. Contact with the solution causes the epithelial cells to lose their adherence at the BMZ, most likely by destroying a portion of that cell population. The epithelium is then raised by pushing the epithelium in a manner similar to striping a wall of paint. The exposed collagenous portion of the corneal stroma is then ablated to reshape its surface. A weakened epithelium is then rolled back into place to serve as a bandage. However, this “bandage” fails to restore the epithelium to its original state, i.e., it does not preserve the integrity of the epithelium, thereby reducing its clarity, impermeability to water, and barrier function. Furthermore, the ability of the epithelium to adhere to the corneal stromal surface is impaired.

Devices and methods of delaminating at least a part of the corneal epithelium have recently been developed. In particular, the epithelium may be partly delaminated, leaving a portion of the separated epithelium attached. The separated epithelium remains viable, and can be re-attached to the cornea or to an ocular device inserted onto the corneal stroma. Examples of delaminating devices and methods may be found in U.S. Pat. No. 6,544,286, U.S. patent application Ser. No. 10/243,121 (filed Sep. 13, 2002), U.S. patent application Ser. No. 10/346,664 (filed Jan. 17, 2003), and U.S. provisional application 60/505,219 (filed Sep. 22, 2003) and 60/580,430 (filed Jun. 16, 2004), which are hereby incorporated by reference in their entirety.

Partial delamination of the epithelium may be useful for implanting an ocular device such as a lens. An ocular device inserter may place an ocular device between the coreal stroma and the epithelium, so that the epithelium (which has been partially delaminated) retains the ocular device securely, even after removal of any ocular device insertion apparatus. An ocular device inserter may therefore place the lens onto the corneal stroma, maintain the integrity of the epithelial layer, and allow the epithelium to retain the lens in position, even after the removal of the epithelial layer.

### REFERENCES

Kiistala, U. (1972). “Dermal-Epidermal Separation. II. External Factors in Suction Blister Formation with Special Reference to the Effect of Temperature,” Ann Clin Res 4(4):236-246.
Azar et al. (2001). “Laser Subepithelial Keratomileusis: Electron Microscopy and Visual Outcomes of Flap Photorefractive Keratectomy,” Curr Opin Ophthalmol 12(4):323-328.
Beerens et al. (1975). “Rapid Regeneration of the Dermal-Epidermal Junction After Partial Separation by Vacuum: An Electron Microscopic Study,” J Invest Dermatol 65(6):513-521.
Willsteed et al. (1991). “An Ultrastructural Comparison of Dermo-Epidermal Separation Techniques,” J Cutan Pathol 18(1):8-12.
Van der Leun et al. (1974). “Repair of Dermal-Epidermal Adherence: A Rapid Process Observed in Experiments on Blistering with Interrupted Suction,” J Invest Dermatol 63(5):397-401.
Katz SI. (1984). “The Epidermal Basement Membrane: Structure, Ontogeny and Role in Disease,” Ciba Found Symp 108:243-259.
Green et al. (1996). “Desmosomes and Hemidesmosomes: Structure and Function of Molecular Components,” FASEB J 10(8):871-881.

None of the cited references shows or suggests my invention as described herein.

### SUMMARY

The description includes ocular device applicators for introducing an ocular device beneath a corneal epithelium. The applicators include an ocular device holder, and a manipulator region. The ocular device holder is configured to place the ocular device beneath the layer of the corneal epithelium upon the cornea, and to hold the ocular device with the applicator until such placement; wherein the ocular device holder is further configured to controllably release the ocular device. The manipulator region is attached to the ocular device holder and is configured to control motion of the ocular device holder.

In some versions of the applicator, the ocular device holder comprises a surface which conforms to at least one surface of an ocular device. In some versions, the ocular device holder comprises a recessed region for holding the ocular device. In some versions, the ocular device holder comprises graspers.

In some versions, the applicator is adapted to couple with a guide. The guide may be configured to assist a user in positioning an ocular device onto a cornea that has been at least partially delaminated. For example, the guide may be a suction ring which attaches to the surface of the eye and can be used to guide both a delaminator and the applicator. Thus, at least a region of the manipulator may be configured to couple with the guide.

In some versions of the applicator described herein, the applicator may also include a force transducer configured to apply force to an ocular device. In particular, the applicator may apply force to an ocular device in or near the applicator holder. In one version, the force transducer is a plunger. In another version, the force transducer is configured to vibrate at least a portion of the applicator holder. In another version, the force transducer applies pressure (e.g. positive or negative pressure) to at least a portion of the ocular device.

In some versions, the manipulator portion of the applicator is configured to comprise a handle, perhaps separable from the device holder or holder region. Further, the applicator manipulator (or manipulator region) may be connected to a driver capable of moving at least a region of the applicator.

The applicator may also be configured to be single-use.

In some versions, at least a portion of the applicator comprises a low-friction surface. Low-friction surfaces (e.g. diamond, polished, lubricated surfaces, etc.) may be more gentle on the eye and the ocular device, particularly the delaminated epithelial layer. The low-friction surface may be a property of the material, or it may comprise a coating (or both).

The applicator may also be configured to receive an ocular device from an ocular device loader.

Also described herein are ocular device applicators for introducing an ocular device beneath a corneal epithelium comprising an ocular device holder, a manipulator (or manipulator region) attached to the ocular device holder, and an ocular device. The applicator is configured to place the ocular device beneath the layer of the corneal epithelium upon the cornea, and to hold the ocular device with the applicator until such placement. The ocular device holder is also configured to controllably release the ocular device. The manipulator is attached to the ocular device holder and is configured to control motion of the ocular device holder. The ocular device may be seated in the ocular device holder of the applicator.

In some versions of the applicator, the ocular device is releasable secured within the ocular device holder. Examples of ocular devices include lenses, filters, and implants.

Also described herein are ocular device applicators for introducing an ocular device beneath a corneal epithelium comprising an ocular device holder and a manipulator having a proximal and a distal end, therein the ocular device holder is connected to the distal region of the manipulator. The ocular device holder is configured to place the ocular device beneath the layer of the corneal epithelium upon the cornea, and to hold the ocular device with the applicator until such placement.

The manipulator has a proximal and a distal end, and the ocular device holder is connected to the distal region of the manipulator.

Also described herein are methods of applying an ocular device to an eye using an ocular device applicator comprising: positioning an ocular device over a region of the cornea from which the epithelium has been at least partially removed, and releasing the ocular device from the applicator. The ocular device is releasably secured within the ocular device applicator which comprises an ocular device holder and a manipulator. In some versions, the method further includes withdrawing the applicator. In some versions, the method also includes replacing the delaminated epithelium over the ocular device.

### BRIEF DESCRIPTION OF THE DRAWINGS

FIG. 1A is a bottom view of an applicator as described herein.

FIG. 1B is a side view of the applicator shown in FIG. 1A.

FIG. 2A is a bottom view of another version of an applicator as described herein.

FIG. 2B is a side view of the applicator shown in FIG. 2A.

FIG. 2C is a bottom view of another version of an applicator as described herein.

FIG. 2D is a side view of the applicator shown in FIG. 2C.

FIG. 3A is a bottom view of an applicator having a force transducer as described herein.

FIG. 3B is a bottom view of another applicator having a force transducer as described herein.

FIG. 4 illustrates an eye with an attached guide and a partially delaminated corneal epithelium.

FIG. 5A is a bottom view of another applicator as described herein.

FIG. 5B is a side view of the applicator of FIG. 5A.

FIG. 5C is a top view of the applicator of FIG. 5A.

FIG. 6 is a bottom view of another applicator as described herein.

### DETAILED DESCRIPTION

The ocular device applicators described herein (also referred to as “applicators”) may be used for inserting ocular devices onto a corneal surface from which the epithelium has been at least partly removed.

A continuous layer of corneal epithelium may be separated from or lifted from the anterior surface of the eye by applying various forces (e.g. mechanical force) to this anterior surface, or to the basal cell layer, or to the junction between the basal cell layer and the Bowman membrane (the “lamina lucida”). The term “continuous” as used herein means “uninterrupted”. More or less epithelium may be separated from the cornea. For example, the devices and methods disclosed herein may be used to utilize or perhaps to create a loose flap of corneal epithelium, leaving less than 50% (preferably between 10% and 50%) of the edge of the delaminated epithelium attached to the cornea. Similarly, a flap of corneal epithelium may be made from the corneal epithelium, leaving between 50% and 75% of the edge of the delaminated epithelium attached to the cornea. A half flap, or tight pocket, of delaminated corneal epithelium may also be formed by leaving between 50% and 95% of the edge of the delaminated epithelium attached to the cornea. The epithelium thus separated may comprise viable (e.g. living) epithelial cells which may later be repositioned back onto the cornea and/or an implanted ocular device using the devices and methods included here.

Described herein are ocular device applicators for introducing an ocular device beneath a previously separated corneal epithelium that are configured to place the ocular device upon the cornea beneath the previously separated layer of the corneal epithelium, and to hold the ocular device until such placement. In some versions, the applicator is configured to introduce the ocular device upon the cornea and beneath a previously separated corneal epithelium pocket. In some versions, the applicator is configured to introduce the ocular device upon the cornea and beneath a previously separated corneal epithelium pocket having a pocket opening by introducing the ocular device through that pocket opening. In some versions, the applicator is configured to introduce the ocular device upon the cornea and beneath a previously separated corneal epithelium pocket having a pocket opening without substantial detriment to the epithelial layer. In some versions, the applicator is further configured to controllably release the ocular device.

Also described herein are applicators that may be used to insert an ocular device onto the region of the cornea that has been delaminated. In particular, the applicators described herein allow an ocular device to be inserted onto the delaminated cornea, beneath the epithelium that was separated from the cornea. The separated epithelium can then be positioned and/or replaced atop the inserted ocular device.

The term “ocular device” is intended to include any implantable ocular device, preferably ocular devices intended to modify, improve or correct vision in a patient in need thereof. One such suitable ocular lens device to be used with the present invention is described in Application No. PCT/US01/22633 which is herein incorporated by reference in its entirety. Examples of ocular devices include: lenses (such as contact lenses, implantable lenses, etc.), filters (e.g. diffraction (line or pinhole) gratings, polarizers, etc.), implants (e.g. implants to reshape the eye surface), and the like.

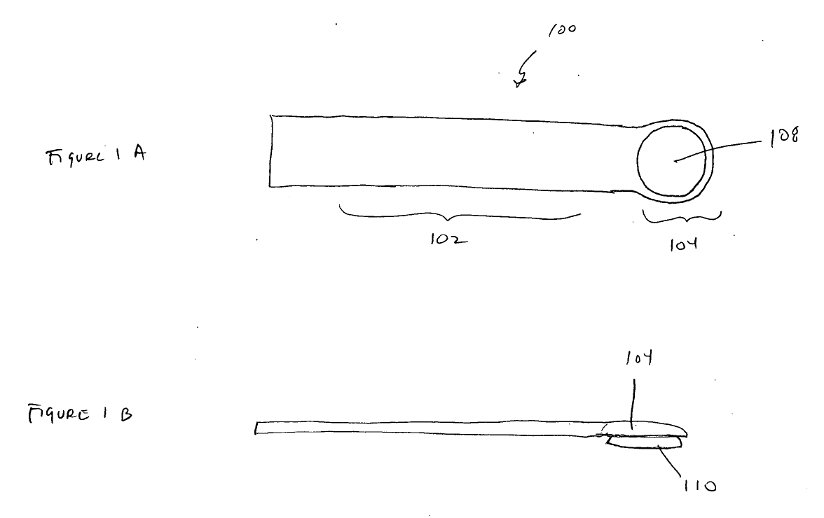

FIGS. 1A and 1B show a first variation of an applicator 100. FIG. 1A shows a bottom view of an applicator having an elongated manipulator 102. A holder 104 is attached to the manipulator at the distal end of the applicator. For convenience, the “bottom” of the applicator refers to the face of the applicator configured to be closest to the corneal stroma when being used to apply an ocular device. Similarly the “top” of the applicator refers to the face of the applicator opposite the bottom face.

The applicators described herein may be of any suitable size configured to achieve the functional results specified herein, particularly sizes in which the holder region may be inserted between a partially delaminated epithelium and the corneal stroma from which it was delaminated. For example, a portion of an applicator, particularly the holder, may be inserted into an epithelial pocket, and thus, may be configured to fit into the pocket without damaging it. FIG. 1B shows a side view of the applicator in FIG. 1A. An example of an ocular device 110 is shown seated in the holder 104.

The applicators described herein may also include one or more force transducers, for applying force to an ocular device to secure it within the holder or to release it from the holder. The applicators may also include a guide interface to aid the user in inserting the ocular device into the eye. Finally, the applicator may be used in conjunction with a loading device (for placing the ocular device into the holder region), and a driver for helping control the motion of the applicator.

Ocular Device Holder

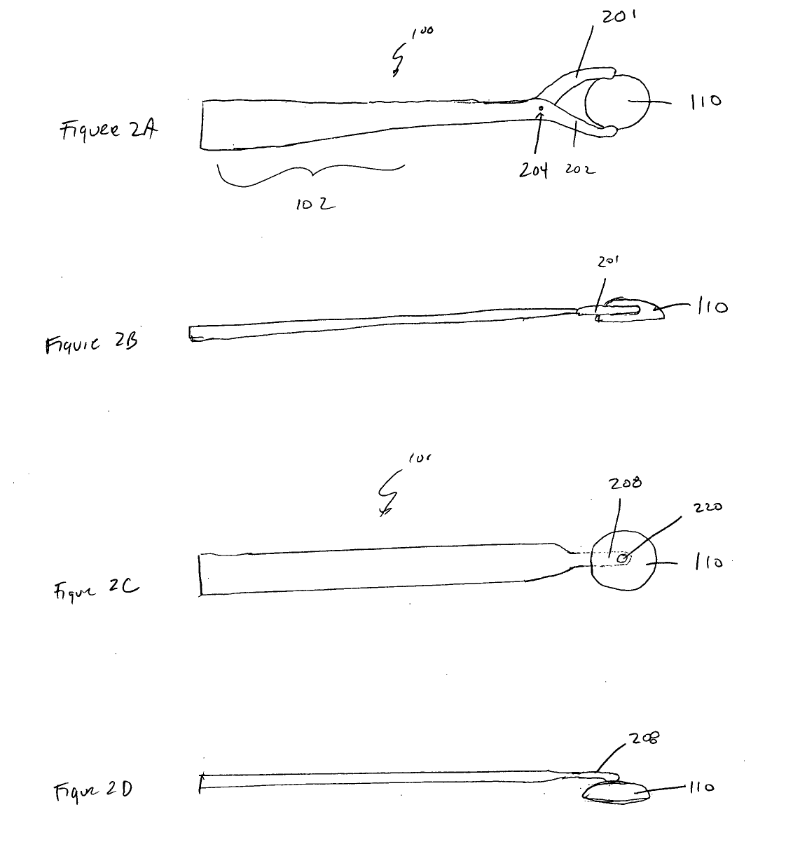

The ocular device holder 104 of the applicator may be of any shape or structure adequate to controllably hold the ocular device so that the ocular device may be positioned over the delaminated cornea and released by the user. FIGS. 1A, 2A and 2C show bottom views of variants of the ocular device holders described herein.

In one version, the ocular device holder is shaped so that at least a portion of the holder conforms in general to the shape of a region of the ocular device. FIG. 1A shows a cup-like holder region, in which the holder has a concavity which matches the convex side of an ocular device (e.g. a lens). Because ocular devices may require oriented placement on the eye, the holder may be configured so that the proper orientation is reflected in the design of the applicator, and, in turn, that orientation is communicated to the user. In FIG. 1A, the concavity into which the ocular device fits 108 allows an ocular device to be placed on the eye so that the concave side of the ocular device fits to the curved surface of the eye.

In some versions, the holder region 104 is smaller than the ocular device which it holds, so that the ocular device 110 projects from the profile of the applicator as shown in FIG. 1B. In some versions, the holder region 104 holds the entire ocular device, so that none of the ocular device projects beyond the profile of the applicator. The holder surface (e.g. the surface contacting at least a portion of the ocular device) may also be textured or discontinuous. For example, the surface of the holder may be grooved, foraminated, etc.

The cup-like ocular device holder shown in FIGS. 1A and 1B supports the ocular device over much of the surface of at least one face of an ocular device. The ocular device holder may hold an ocular device by contacting a much smaller (or larger) portion of the surface of an ocular device. Other variations of the applicator incorporate holders which have only minimal contact with the surface of the ocular device. For example, rather than a complete concavity as shown in FIGS. 1A and 1B, the holder may comprise a partial cavity. For example, the holder may support the ocular device on two or more fork-like projections (tines). These projections may be curved (e.g. concave). In another version, the holder contacts only a portion of an ocular device. For example, the holder may comprise just the proximal side of the holder shown in FIG. 1A. In some versions it may be desirable to minimize the size of the applicator holder to allow the user to more easily observe the placement of an ocular device when using the applicator. Reducing the size of the holder and/or nearby portions of the manipulator reduces the potential that the applicator will contact the delaminated epithelium or other parts of the eye.

In one version of the ocular device holder, the holder contains one or more graspers for releasably securing an ocular device. FIG. 2A shows a version of the applicator in which the ocular device holder comprises one or more hinged tines 201,202 which contact two regions of the ocular device. A side perspective view of this applicator is shown in FIG. 2B. The tines of the holder may apply pressure to the ocular device and thereby secure the ocular device. Pressure is applied by moving the tines closer to each other. In FIG. 2A, the upper tine 201 is hinged 204 so that it may move relative to the lower tine 202. The movable (upper) tine may be controlled by the user, allowing the user to controllably grasp and release an ocular device. Graspers (e.g. the tines of FIGS. 2A and 2B) may be oriented in any way so that the ocular device may be releasably secured without being damaged. For example, the ocular device shown in FIG. 2A is presumably rigid enough so that the securing pressure from the holder does not damage or collapse the ocular device, making it difficult to apply. An ocular device could be grasped in any way that would allow the device to be secured and placed onto the eye.

A single tine may also be used as a holder to secure an ocular device. FIG. 2C shows a view of an applicator in which the holder is a single tine 208 to which an ocular device 110 is attached. The ocular device may be secured to the single tine by the application of a force (e.g. suction applied thorough a suction port 220 as shown in FIG. 2C), or by any other releasable attachment means. For example, the ocular device may be secured to the holder by an adhesive material which maybe controllably dissolved or removed once the ocular device is properly positioned. The applicator shown in FIG. 2C has an ocular device holder that is smaller than other ocular device holders shown in FIGS. 1A and 1B. The small holder 208 secures the ocular device while minimizing the potential surface area which may contact the eye when used to apply an ocular device.

The holder may be other shapes, or combinations of shapes. For example, the ocular device holder may comprise a concavity and also an additional member (or concavity) which can enclose a portion of the ocular device. Thus, the holder may surround the ocular device on two or more sides. The ocular device is released by “opening” the holder.

An ocular device may also be held in an applicator holder differently than illustrated here. For example, the holder may secure an ocular device only at one edge, allowing the ocular device to project away from the applicator.

A designer may determine the shape of the holder region of the applicator based on the shape and composition of an ocular device. For example, ocular devices having concave and convex surfaces (as shown in FIGS. 1A, 2A and 2C) may work best with holders having a concave surface. Similarly, holders providing more support surfaces (e.g. the cup-like holders) may be preferable for ocular devices made of less rigid materials.

In some versions, at least a portion of the ocular device applicator has a low-friction surface. In particular, surfaces of the applicator which may come into contact with the delaminated epithelium. The applicator may be inserted beneath a flap or into a pocket of delaminated epithelium when placing the ocular device onto the delaminated corneal region. Thus, the upper surface of the applicator (the surface furthest from the surface of the cornea) may contact the living, yet delaminated epithelium. Preventing the applicator from sticking to the delaminated epithelium may prevent damage to the epithelium and may also make it easier to operate the applicator. It may also be beneficial for the surfaces of the holder to be low-frictional surfaces, helping prevent damage to ocular devices held therein.

In one version, at least a region of the applicator is coated with a material providing low-friction properties, e.g. para-xylyene polymers (such as parylene C, parylene N, and parylene D), polyfluorocarbon polymers (such as PTFE, FEP, and similar materials). In one version, at least a region of the applicator is polished to reduce friction. In version the surfaces likely to contact the eye (in particular the delaminated epithelium) comprise low-friction surfaces.

Similarly, the applicator surfaces which may come into contact with the eye during use may be substantially blunt in order to avoid damage to the eye. The applicator may also incorporate one or more therapeutic agents. For example, the applicator may be coated with a therapeutic agent (e.g. an antibiotic, anticoagulant, growth factor, etc.).

Any of the applicators described herein may include a force transducer for securing and/or releasing an ocular device from the holder. In one version, a force transducer applies pressure to secure and/or release an ocular device from the holder. For example, in one version, the ocular device applies negative pressure (vacuum) to secure the ocular device in the holder, and positive pressure to release the ocular device once it is in position over the eye. Pressure may be applied by gas (e.g. air pressure) or by liquid (e.g. water or saline), or by solid (e.g. a plunger-type mechanism). The holder may also be adapted to house a force transducer.

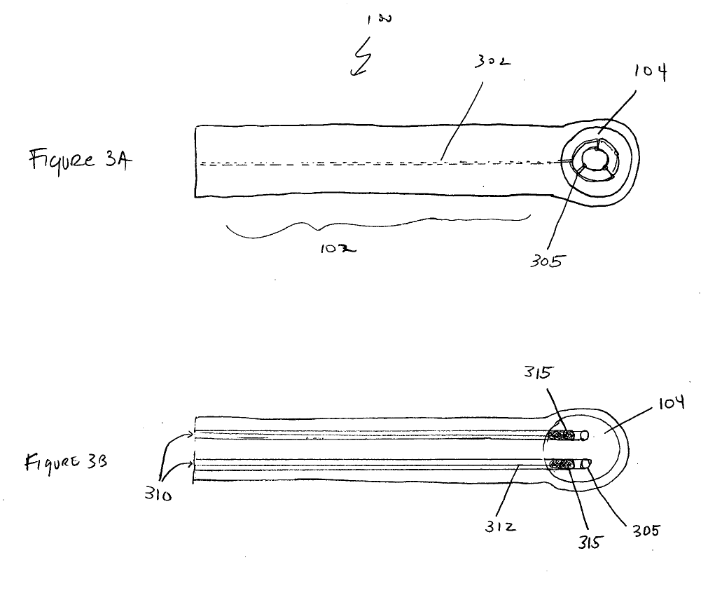

FIG. 3A shows an example of an applicator 100 in which the holder 104 and the manipulator 102 have been adapted to apply pressure to secure and/or release an ocular device. A gas or liquid may be applied or withdrawn though a channel 302 (or channels) in the manipulator; this channel is in fluid connection with inlet/outlet ports 305 in the holder. Thus, force may be applied to an ocular device in the holder though the inlet/outlet ports. Force from the force transducer may also be used to load an ocular device into the applicator.

In one version, an ocular device is held in the applicator holder by applying a vacuum. One or more channels 302 connect to the holder as shown in FIG. 3A. Force is applied to an ocular device in the holder through openings 305 in the holder that connect to the channel. Negative pressure is applied to secure the ocular device in the holder (e.g. by drawing a vacuum) and positive force is applied to release the ocular device from the holder. For example, air pressure (e.g. from air or any other gas) may be applied through the channel to release the ocular device. In another example, fluid pressure (e.g. from water or saline pushed through the channel) is applied to release the ocular device. Any fluid could be used to controllably hold and release the ocular lens in the applicator. Further, the channel may be used to apply useful substances (e.g. liquids such as saline, medicaments, etc.).

FIG. 3B shows an example of an applicator with a force transducer that uses a plunger-type mechanism. In FIG. 3B two plunger-type force transducers 312 are located in channels 310. Each plunger comprises a stiff, elongated member. An ocular device can be released from the holder by moving the plungers forward, resulting in the end of a plunger pushing against an ocular device seated in the holder. In one version, one or more plungers 312 also have an endpiece 315 which fits snugly within the channel 310. A negative pressure (e.g. vacuum) may be created within the plunger channel 310 by withdrawing the endpiece into the channel. This negative pressure may secure the ocular device in the holder.

In another version of the force transducer, a plunger-type mechanism is used to secure an ocular device within the holder by securing the ocular device between the end of a plunger and another region of the holder. The ocular device may be released by withdrawing the plunger. The grasping-type holder shown in FIG. 2A and 2B is another type of force transducer useful for securing ocular devices in the holder and releasing ocular devices from the holder.

Force may be indirectly applied to an ocular device held by the applicator. Vibrational energy may be applied to release the ocular device using a force transducer. For example, the holder (or a region of the holder) may be oscillated (e.g. using audiable sonic or ultrasonic/supersonic frequencies) by a force transducer comprising a driver configured to oscillate the holder. The entire applicator may be vibrated, or a portion of the applicator. In some versions, only the holder is vibrated. In other versions, the manipulator is also oscillated. The frequency and extent of the vibration may be controlled by the user, or may be automated. In one version, an ocular device is released from the applicator by briefly vibrating the holder. Vibrating the holder may disrupt any surface tension holding an ocular device in the holder, releasing the ocular device from the applicator.

The applicator may incorporate one or more transducers capable of applying virtually any kind of energy (e.g. force) useful for securing and/or releasing an ocular device from the applicator. Examples of transducers include transducers configured to emit thermal energy, magnetic energy, sonic energy, electromagnetic energy, etc. Furthermore combinations of transducers, or transducers configured to apply different kinds of energy (including force), may also be incorporated into the applicator.

An ocular device may be passively secured in the holder, in addition to or instead of being secured by active methods (e.g. mechanically securing the ocular device, or securing the ocular device by applying a vacuum). For example, ocular devices may be passively secured in the applicator holder by using adhesives, surface tension, dehydration, etc.

In one version, the ocular device is secured in the holder by a releasable adhesive. In particular, a dissolvable adhesive may be used. For example, in one version a water-soluble material secures the ocular device in the holder until it is ready to be released after insertion. Examples of water-soluble materials include, but are not limited to: polymers such as polyvinylalcohol, biopolymer such as hyaluronic acid (HA), and polysaccharides. Application of a fluid that releases the adhesive (e.g., saline or other beneficial fluid) causes the adhesive to dissolve or otherwise release, thereby releasing the ocular device. Fluid may be applied locally (e.g. through a channel 302) or over a larger area of the cornea.

Other variations of the applicator may secure and/or release ocular devices from the holder using any combination of the devices, transducers and techniques discussed above.

Manipulator Region

The applicator may also comprise a manipulator region 102 as shown in FIGS. 1-4. In FIGS. 1A and 1B, the manipulator region 102 is shown as an extended flattened region connected to the holder 104. In general, the manipulator region may be any shape which allows the user to manipulate the applicator. The manipulator generally allows the user to move the applicator (e.g. to position the applicator over the appropriate region of the cornea). The manipulator may also incorporate a control or controls for releasing and/or securing an ocular device in the applicator holder. For example, the manipulator may include a port for applying or withdrawing fluids to and from channels 302 in the applicator. In FIGS. 1A and 1B the holder is configured as a cavity in the manipulator region.

The manipulator may be configured to give stability to the applicator in space. In one version, the manipulator is an elongated stiff member connecting to the holder; movement of the manipulator results in movement of the holder and therefore of the ocular device. The manipulator may be configured for manual control by the user. In one version, the manipulator is configured as a handle. A user may grasp the handle and manually move the applicator into position: to load an applicator with an ocular device; to position an ocular device between a delaminated epithelium and the underlying corneal stroma; to release an ocular device onto the cornea; and to withdraw the applicator. The manipulator may also comprise a gripping surface and/or controls such as a trigger to release the ocular device.

In one version, the applicator manipulator is configured to attach to an automated or mechanical controller. For example, the manipulator may include attachment sites for attachment to a x,y stage or other translational machinery.

In one version, the applicator manipulator is adapted to incorporate a transducer. For example, FIGS. 3A and 3B show manipulators comprising channels though which force is applied to an ocular device in the holder (e.g. for a plunger or the movement of a fluid). The applicator may also comprise controls for the force transducer, or the controls may be separate from the manipulator portion of the applicator.

The manipulator may be of any size (e.g. length, thickness, shape, etc.) convenient for controlling the applicator. For example, the manipulator may be adequately long so that it could be grasped and controlled by a typical user. In one version, the applicator is an elongated member having a proximal end and a distal end; the holder region is attached to the applicator at the distal end. The manipulator may be configured so that the holder readily reaches the region of the eye where the ocular device is to be positioned. For example, the manipulator may be narrower in the region nearest to the applicator holder to prevent the holder from interfering contacting the delaminated epithelium or eye surface.

Methods of Use

The applicators described herein may be used to load the applicator with an ocular device, and/or to position an ocular device relative to an eye (particularly an eye from which the epithelium has been at least partially delaminated), and/or to place the ocular device onto the eye (e.g. between the corneal stroma and the epithelium), and/or to reposition the ocular device on the eye, and/or to withdraw the applicator from the eye.

Loading the Applicator

The applicator may further include an ocular device that is to be applied to an eye. The ocular device may be pre-loaded into the applicator (e.g. at the time of packaging) or may be loaded by a user or other intermediary. Preloaded applicators may be packaged as individual applicators with ocular devices already in the applicator holder. For example, applicators and ocular devices may come as a package of one or more devices which could be individually sealed and sterilized (e.g. as a disposable packet). In one version, the applicator is configured to be disposable and/or single-use. For example, the applicator be designed to allow the ocular device to be released only once. Single-use applicators may be made of inexpensive (e.g. less durable) materials, and may avoid problems with later sterilization. In another version, the applicator may be configured for multiple uses.

The applicator may be loadable, meaning that an ocular device may be loaded into the holder of the ocular device before using the applicator to apply an ocular device to an eye. Loadable applicators may be configured for single use or multiple uses. Multiple-use applicators may be fabricated of a material (or materials) which are sterilizable.

Applicators may be manually loadable, loaded with the assistance of accessory devices, or loaded automatically. An applicator is manually loaded by placing the ocular device in the correct orientation in the holder of the applicator. The ocular device may then be secured into the holder. Ocular devices may be kept (e.g. for storage or shipment) in solutions such as saline, or may be kept dry. For example, when the ocular device is a hydrated lens, the ocular device may be transferred from storage in a saline solution into the holder of the applicator by the manual efforts of a user. In some versions of the applicator, a force transducer may be used to assist loading. For example, in applicators in which an ocular device is secured into the holder by suction, using the force transducer to apply suction while loading the ocular device may facilitate loading, particular in ocular devices suspended in fluids.

Additional devices may also be used to load the applicator. In one version, the ocular device is processed by an intermediary device which may orient the ocular device, prepare it for loading into the applicator and position the ocular device for transfer. In one version, the applicator may be adapted to be used in conjunction with an intermediary loading device. The loading device may draw the ocular device onto the holder of the applicator (e.g. by suction, by centrifugation, by sieving the storage medium, by vibration, etc.). In one version, the ocular device is stored in container and kept in a solution (e.g. saline); the applicator may be attached to the terminus of a funnel-shaped device into which the solution containing an ocular device is poured. The fluid may be drawn over applicator holder region at the end of the funnel-shaped structure. In one version, fluid is suctioned from the funnel-shaped device by the force transducer of the applicator. In this version, an ocular device stored in this fluid would eventually settle into the holder of the applicator. If the ocular device is properly oriented in an applicator holder in which the holder conforms to a specifically oriented surface of the ocular device, fluid will stop being drained through the force transducer once the ocular device is properly seated in the holder. For example, the ocular device may be a lens having a concave side, and the applicator holder may be a concavity as shown in FIG. 1A.

Once the ocular device is seated in the applicator holder, the ocular device may be secured into applicator holder. In some versions this may mean that the force transducer is used to apply a force securing the ocular device into the holder.

Positioning the Ocular Device

The applicator and loaded ocular device may be applied to an eye. Preferably an eye from which one or more portions of the epithelium have been delaminated from the corneal stroma as described in U.S. Pat. No. 6,544,286, U.S. patent application Ser. No. 10/243,121 (filed Sep. 13, 2002), U.S. patent application Ser. No. 10/346,664 (filed Jan. 17, 2003), and U.S. provisional application 60/505,219 (filed Sep. 22, 2003) and 60/580,430 (filed Jun. 16, 2004), the entirety of which are incorporated by reference in their entirety. The applicator may also be used to apply ocular devices to eyes which have not been delaminated.

The applicator may apply an ocular device between the corneal epithelium and the corneal stroma, and onto the corneal stroma. In one version, the epithelium has been peeled back (e.g. a flap of epithelium has been separated from the cornea). In one version, the epithelium has been delaminated, but remains on top of the cornea. In another version, the epithelium has been separated from the epithelium but remains as a ‘pocket’ above the epithelium. In every case, the applicator may be used to insert the ocular device onto the de-epithelized corneal stroma.

A user may apply the ocular device onto the corneal stroma by a “freehand” technique. For example, the user guides the applicator into position between the delaminated epithelium and the corneal stroma without using any additional structural guide. Alternatively, a user may apply the ocular device using an additional guide. Thus, the applicator may also be adapted to couple to a guide for insertion of the ocular device.

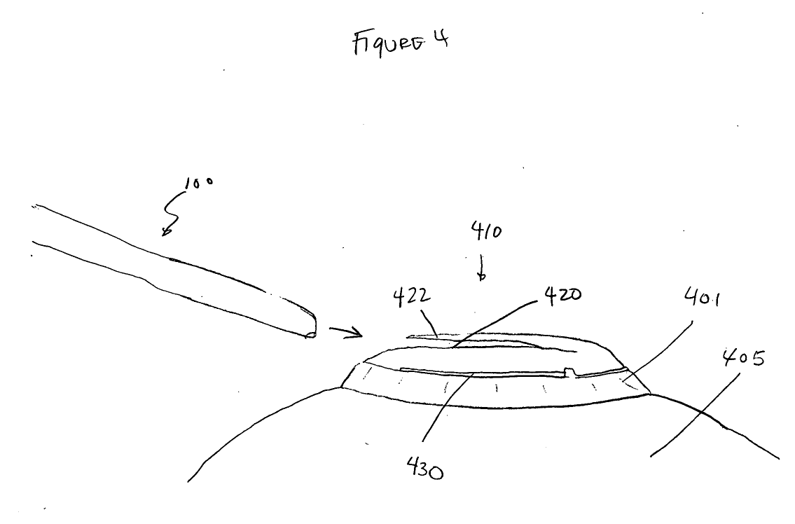

In particular, applicators may be adapted to couple to the same guides used to delaminate the epithelium. For example, FIG. 4 shows a suction ring 401 on the surface of an eye 405 that has been delaminated 410. The suction ring is one of a class of devices which may secure and/or present the surface of the eye so that it may be operated upon (e.g. delaminated). Any appropriate device for presenting the surface of the eye may be used. In FIG. 4, the suction device is placed onto the eye, and secured in position. This process allows reproducible access to a region of the eye surface 410, which may be delaminated as shown. The suction device shown in FIG. 4 also has a track-like region 412 on one or more regions of the perimeter of the suction device, allowing a device such as a delaminator or the inserter to move in a predetermined fashion across the surface of the eye. In this example, an applicator 100 is shown to the left of the eye, preparing to insert an ocular device between the epithelial region 422 and the corneal surface 420. This applicator is configured to fit into the track region 430 of the guide device (here a suction ring), guiding the applicator into position so that it can accurately apply an ocular device.

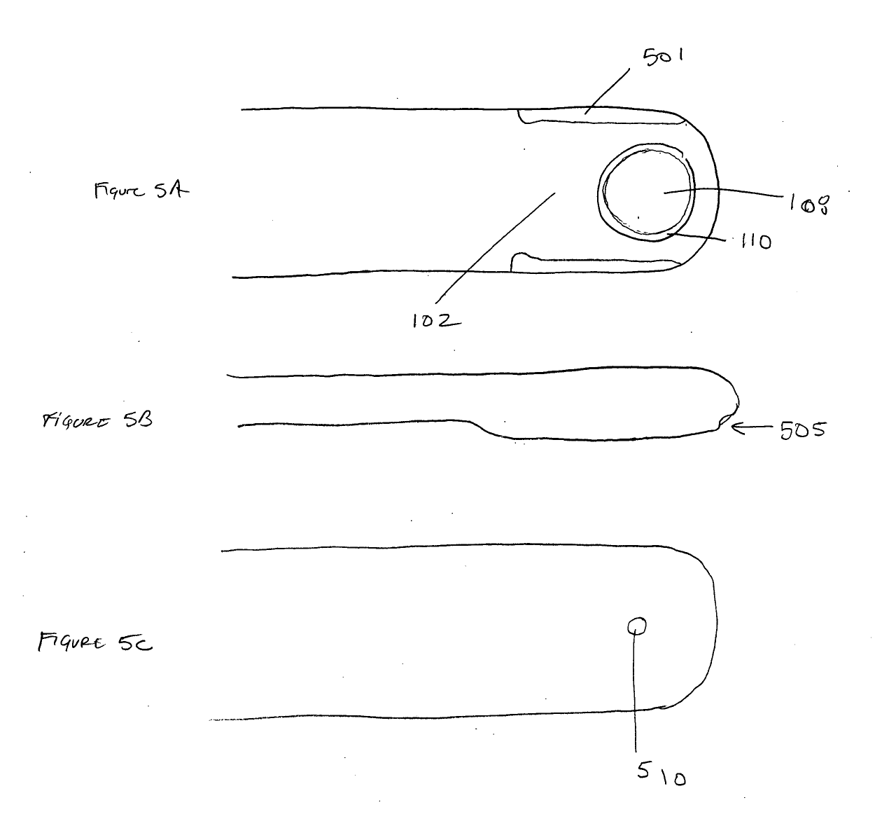

FIGS. 5A-5C show an applicator which has been adapted to fit into a guide similar to that shown in FIG. 4. FIG. 5A shows a bottom view of an applicator having a recessed track 501 on either side of the ocular device holder which is configured to couple to a guide attached to an eye. Additional structure may be included to connect the guide to the applicator, or to prohibit undesirable motion by the applicator once it contacts the guide. The applicator in FIG. 5A has a holder 108 which is a cup-like concavity as in FIG. 1A; the holder is surrounded by the manipulator 102 region. FIG. 5B shows a profile of the applicator of FIG. 5A. The applicator of FIGS. 5A-5C completely surrounds the ocular device 110 secured in the holder 108, so that the lower region of the applicator 505 in FIG. 5B may connect to a guide as shown in FIG. 4. FIG. 5C shows a top view of the applicator. The applicator is shown with a window 510 through the applicator, which may help a user when positioning the ocular device with the applicator. Windows of any shape and size may be incorporated into the applicator. In FIG. 5C, the window is shown at approximately the center of the holder, corresponding to the center for an ocular device secured into the holder.

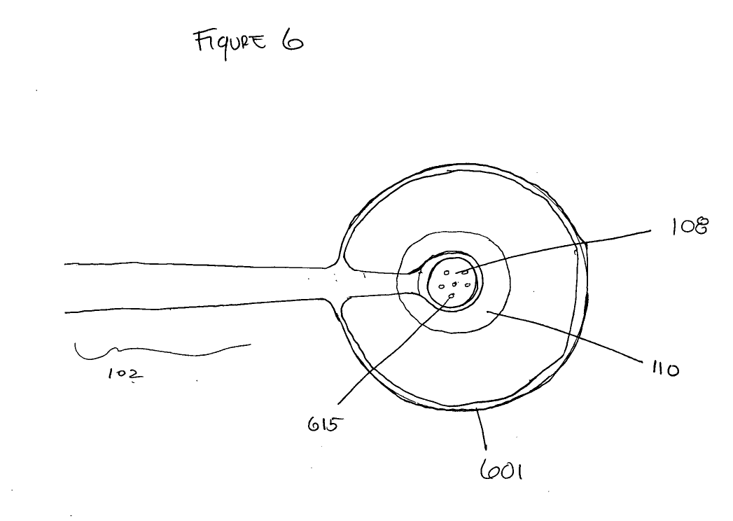

FIG. 6 shows another variation of an applicator adapted to be used with a guide. In FIG. 6 the manipulator comprises a guide-coupling region 601 that is shown as a loop. The guide-coupling region fits into a complimentary region in a guide device. Other shapes and styles of guide-coupling regions may be used. The applicator in FIG. 6 has a small holder 108 which is shown securing an ocular device 110 by means of a five suction ports 615.

Once the applicator is in (or approximately in) a desired position over the eye, the ocular device may be released from the applicator onto the surface of the corneal stroma. As described, the applicator may release the ocular device by removing the force securing the ocular device in the applicator holder (or by removing the passive material securing the ocular device), allowing the ocular device to drop onto the corneal stroma. The applicator may also be configured to apply force to release the ocular device. For example, the force transducer of the ocular device may apply force (e.g. pressure) to release the ocular device from the holder onto the eye.

Once the ocular device has been placed on the eye, the applicator may also be used to more precisely move or reposition the ocular device. In some versions, the ocular device may be reloaded onto the applicator (e.g. by reapplying a negative pressure, or by grasping the ocular device).

In some versions of the applicator, one or more portions of the applicator may be adapted to “nudge” or move an ocular device which has been placed onto the eye. For example, an edge of the applicator could be configured to touch the ocular device and/or the eye without harming them. In one version, the edge of the applicator is dull and has a low-friction coating.

Some versions of the applicator may comprise a repositioning member for small (e.g. fine) movements of the ocular device on the corneal stroma. In one version, a repositioning member may be a retractably member that projects from the distal tip of the applicator (e.g. near the holder); when extended, this region can be used to nudge an ocular device after it has been released from the applicator. In the retracted position, the repositioning member does not interfere with motion of the applicator across the eye. Retractable repositioning members are particularly useful in versions of the applicator adapted to be used with a guide, since these applicators may be configured so that they would otherwise withdraw from the guide without interfering with the released ocular device on the surface of the corneal stroma.

In general, once the applicator has released the ocular device, which is place in a desired position, the applicator may be withdrawn. In some cases, the epithelial flap is then placed over at least one surface of the ocular device.

The structure and physiologic properties for my invention, as well as certain of the benefits particular to the specific variations of this applicator device, have been described. This manner of describing the invention should not, however, be taken as limiting the scope of the invention in any way.

We claim:

1. An ocular device applicator for introducing an ocular device beneath a previously separated corneal epithelium, said applicator configured to place the ocular device upon the cornea beneath the previously separated layer of the corneal epithelium, and to hold the ocular device until such placement.

2. The applicator of claim 1 configured to introduce the ocular device upon the cornea and beneath a previously separated corneal epithelium pocket.

3. The applicator of claim 1 configured to introduce the ocular device upon the cornea and beneath a previously separated corneal epithelium pocket having a pocket opening by introducing the ocular device through that pocket opening.

4. The applicator of claim 3 configured to introduce the ocular device upon the cornea and beneath a previously separated corneal epithelium pocket having a pocket opening without substantial detriment to the epithelial layer.

5. The applicator of claim 1 further configured to controllably release the ocular device.

6. The applicator of claim 5 further configured for removal from beneath the epithelial layer without substantial detriment to the epithelial layer.

7. The applicator of claim 6 configured to introduce the ocular device upon the cornea and beneath a previously separated corneal epithelium pocket having a pocket opening without substantial detriment to the epithelial layer.

8. The applicator of claim 1 further comprising the ocular device.

9. The applicator of claim 8 wherein the ocular device is releasable secured within the applicator.

10. The applicator of claim 9 wherein the ocular device comprises a lens.

11. An ocular device applicator for introducing an ocular device beneath a corneal epithelium comprising:
an ocular device holder configured to place the ocular device beneath the layer of the corneal epithelium upon the cornea, and to hold the ocular device with the applicator until such placement; wherein the ocular device holder is further configured to controllably release the ocular device; and
a manipulator attached to the ocular device holder and configured to control motion of the ocular device holder.

2. The applicator of claim 11 wherein the ocular device holder comprises a surface which conforms to at least a portion of at least one surface of the ocular device.

13. The applicator of claim 11 wherein the ocular device holder comprises a recessed region for holding the ocular device.

14. The applicator of claim 11 wherein the ocular device holder comprises graspers.

15. The applicator of claim 11 wherein the applicator is further adapted to couple with a guide configured to assist a user in positioning an ocular device onto a cornea that has been at least partially delaminated.

16. The applicator of claim 15 wherein the guide is a suction ring.

17. The applicator of claim 15 wherein at least a region of the manipulator is configured to couple with the guide.

18. The applicator of claim 11 wherein the applicator further comprises a force transducer configured to apply force to an ocular device.

19. The applicator of claim 18 wherein the ocular device holder comprises a port configured to connect to a force transducer.

20. The applicator of claim 18 wherein the force transducer is a plunger.

21. The applicator of claim 18 wherein the force transducer is configured to vibrate at least a portion of the holder.

22. The applicator of claim 18 wherein the force transducer applies pressure.

23. The applicator of claim 22 wherein the force transducer applies positive or negative pressure to at least a portion of an ocular device.

24. The applicator of claim 21 wherein the manipulator is configured as a handle.

25. The applicator of claim 21 wherein the manipulator is configured to connect to a driver capable of moving at least a region of the applicator.

26. The applicator of claim 21 wherein the applicator is further configured to be single-use.

27. The applicator of claim 21 wherein at least a portion of the applicator comprises a low-friction surface.

28. The applicator of claim 27 wherein the low-friction surface comprises a coating.

29. The applicator of claim 11 further configured to receive an ocular device from an ocular device loader.

30. An ocular device applicator for introducing an ocular device beneath a corneal epithelium comprising:
an ocular device holder configured to place the ocular device beneath the layer of the corneal epithelium upon the cornea, and to hold the ocular device with the applicator until such placement; wherein the ocular device holder is further configured to controllably release the ocular device;
a manipulator attached to the ocular device holder and configured to control motion of the ocular device holder; and
an ocular device.

31. The applicator of claim 30 wherein the ocular device is releasable secured within the ocular device holder.

32. The applicator of claim 30 wherein the ocular device is a lens.

33. An ocular device applicator for introducing an ocular device beneath a corneal epithelium comprising:
an ocular device holder configured to place the ocular device beneath the layer of the corneal epithelium upon the cornea, and to hold the ocular device with the applicator until such placement; and
a manipulator having a proximal and a distal end, wherein the ocular device holder is connected to the distal region of the manipulator.

34. A method of applying an ocular device to an eye using an ocular device applicator comprising:
positioning an ocular device over a region of the cornea from which the epithelium has been at least partially removed; wherein the ocular device is releasably secured within an ocular device applicator comprising an ocular device holder and a manipulator; and
releasing the ocular device from the applicator.

35. The method of claim 34 further comprising withdrawing the applicator.

36. The method of claim 34 further comprising replacing the delaminated epithelium over the ocular device.
